(19) United States
(12) Patent Application Publication
(10) Pub. No.: US 2004/0241185 A1
(43) Pub. Date: Dec. 2, 2004

(54) Antiviral formulation from Indian seaweeds

(75) Inventors: Anil Chatterji, Goa (IN); V. K. Dhargalkar, Goa (IN); P. K. Syeekumar, Goa (IN); Savita Kotnala, Goa (IN); Reena Rodrigues, Goa (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/842,968

(22) Filed: May 6, 2004

Related U.S. Application Data
(60) Provisional application No. 60/468,466, filed on May 7, 2003.

(51) Int. Cl. A61K 35/80

(52) U.S. Cl. 424/195.17

(57) ABSTRACT

An extract with antiviral activity including a mixture of extracts from Spatoglossum asperum., Padina tetrastromatica, Sargassum tenerrimum, and Stoechospermum marginatum. Also, a process for preparation of the extracts, and a method of treatment using the extracts.

The present invention relates to an antiviral formulation. More particularly, the present invention relates to an antiviral formulation comprising of the extract prepared from the Indian seaweeds. The present invention also relates to a process for the preparation of an antiviral extract from Indian seaweeds. The present invention also relates to an antiviral activity in the extract prepared from the Indian seaweeds. The extract is useful as an antiviral drug for human as well as animals. The extracts prepared from the Indian seaweeds such as Spatoglossum asperum J. Ag., Padina tetrastromatica Hauck, Sargassum tenerrimum J. Ag. and Stoechospermum marginatum (Ag.) Kuetz have been found to be active against two influenza viral subtypes A and B.

Seaweeds are the group of marine plants classified on the basis of pigmentation into green, brown and red algae. In India, seaweeds resources are quire rich. Maximum species of seaweeds are found in Tamil Nadu (302 species) followed by 202 species in Gujarat, 159 in Maharashtra, 89 in Lakshadweep group of Islands and 82 species in Goa. Seaweeds are considered to be an important source of cheap food in some Asian countries where they are consumed as a traditional food. In countries like Malaysia, Indonesia, Thailand, Korea, Japan and Australia seaweeds are used in salads, soups and jelly. The vitamin contents (A, B & B₁₂) are relatively higher in seaweeds as compared to wheat and milk. It has been found that using seaweeds as feed additive increased fertility and birth rate in animals. In spite of rich natural resources in India, seaweeds consumption is relatively is relatively very low.

Besides vast applications of seaweeds as food, feed additive and manure, they are also found to be an important source of several products of pharmacological importance (Bastlow, 1969, Hoppe et al., 1979). These plants are also useful for industrial production of agar, alginate and carrangeenan (Deshmukhe et al, 2001).

The process of preparation of extract from the marine bivalves was developed for the first time by the Russian scientists. A patent on the process developed by Russian scientists was also filed (Patent No. RU 2043109). The applicants herein have also filed patent applications for process of preparation of pharmacologically active extract from the marine bivalves.

Reference is made to a publication wherein aqueous extract prepared from the marine red algae showed antiviral activities against retrovirus, Herpes simplex and HIV (Michael, 1988).

Reference is made to an another publication wherein extract prepared from the marine plants such as seaweed, sea grass and mangroves showed antiviral activities when the extract was tested with Indian strains of Newcastle disease virus (NDV), vaccina virus (VV), encephalomyocarditis virus (EMCV) and Semliki Forest virus (SFA) for in vitro studies (Premnathan et al., 1992).

Reference is also made to an another publication wherein extract prepared from seaweeds from British Columbia showed antiviral activities represented by a common ingredient—the carbohydrate (Hudson, et al., 1999).

The main object of the present invention is to study an antiviral activity in the extract prepared from the Indian seaweeds.

Another object of the present invention is to provide extract from Indian seaweeds such as Spatoglossum asperum., Padina tetrastromatica, Sargassum tenerrimum and Stoechospermum marginatum having high antiviral activity.

Accordingly, the present invention provides an extract showing antiviral activity which comprises collection of four commercially important species of seaweeds such as Spatoglossum asperum., Padina tetrastromatica, Sargassum tenerrimum and Stoechospermum marginatum from the coastal water of Goa, cleaning the weeds with filtered seawater (<5 micron), grinding the seaweeds in a mixer with double distilled water to make a thick paste, lypholization of paste for 8 hours, dissolving 500 g of lypholized seaweeds in 125 ml of double distilled water in a double necked round bottom distillation flask, addition of 10 g dried protosubtiline enzyme, fermentation of mixture at a constant temperature of 40° C. for 2 hours, preparation of active extract by adding 175 ml of Conc HCl, digesting the material for 20 hours at a constant temperature of 100±20° C. on a heating mantle, achieving 5.6 pH of the solution by adding sodium hydroxide, isolating the active extract by keeping the resultant solution in a separating flask for 10 days and carefully removing the middle part of the solution after the formation of different layers.

In one embodiment of the present invention, the cleaning of the weeds was done with filtered seawater (<5 micron).

In another embodiment of the present invention, the grinding of the seaweeds was done in a mixer with double distilled water for making a thick paste.

In yet another embodiment of the present invention, the lypholization of the mixture was done by a table top freeze drying unit for 8 hours.

In yet another embodiment of the present invention, the active extract was prepared by dissolving 500 g of lypholized seaweed in 125 ml of double distilled water in a double necked round bottom distillation flask.

In yet another embodiment of the present invention, the active extract was prepared by adding 10 g dried protosubtiline enzyme and fermenting it at a constant temperature of 40° C. for 2 hours.

In yet another embodiment of the present invention, the active extract was prepared by adding 175 ml of Conc HCl.

In yet another embodiment of the present invention, the digestion of the material was done for 20 hours at a constant temperature of 100±2° C. on a heating mantle.

In yet another embodiment of the present invention, the pH of the solution was achieved to 5.6 pH by adding sodium hydroxide.

In yet another embodiment of the present invention, the active extract was isolated by keeping the resultant extract in a separating flask for 10 days and carefully removing the middle part of the solution after the formation of different layers.

In still another embodiment of the present invention the digestion and distillation of the fermented solution is done with concentrated hydrochloric acid (12-15% of the total weight) at a temperature of 90-105° C. for a period ranging between 12 and 20 hours.

In yet another embodiment of the present invention the particular pH of the extract is achieved using alkali such as NaOH, KOH and NH₃OH etc after cooling the resultant solution at room temperature.

In yet another embodiment of the present invention, the active extract is isolated by keeping the solution in a separating flask for 10-15 days and carefully removing the middle part of the solution after formation of different layers. The detailed steps of preparation of extract are:

1) Collection and preparation of samples: Fresh seaweeds such as Spatoglossum asperum, Padina tetrastromatica, Sargassum tenerrimum and Stoechospermum marginatum we directly collected the intertidal region of coastal waters of Goa. The seaweeds at first we cleaned with filtered seawater (<5 micron), and then ground in the mixer with doubled distilled water to make a thick paste. The thick paste was then freeze dried (lypholized) by a table top freeze drying for 8 hours.

2) Fermentation: 500 g of lypholized seaweed was dissolved in 125 ml of double distilled water in a double necked round bottom distillation flask. 10 g of dried protosubtiline enzyme was added and fermentation of mixture was done at a constant temperature of 40° C. for 2 hours.

3) Digestion and distillation of fermented solution: 175 ml of Conc HCl was added into the double necked round bottom distillation flask containing the resultant solution in the form of thick paste and then the material was digested for 20 hours at a constant temperature of 100±2° C. on a heating mantle.

4) Cooling of the solution: After completion of the digestion and distillation processes, the resultant solution was allowed to cool to room temperature.

5) Maintenance of required pH of the solution: The pH of the solution was maintained at 5.6 by adding sodium hydroxide.

6) Isolation of active extract: The active extract was isolated by keeping the resultant extract in a separating flask for 10 days and carefully removing the middle part of the solution after the formation of different layers.

The aforesaid process requires absolute precaution for pyrogen contamination at all processing steps. All apparatus and reagents must, therefore, be pyrogen free.

The novelty and inventive steps of the present invention is based on fermentation of the seaweed paste with an enzyme-protosubtiline for 2 hours at 40° C. which helps the separation of the active and stable antiviral principals by the enzyme-acid hydrolyzing process.

The following examples are given by way of illustration of the present invention and therefore, should not be construed to limit the scope of the present invention.

### EXAMPLE 1

### Assessment of Antiviral Activity of Extract Prepared from Indian Seaweeds (In Vitro)

The antiviral activity of the extract prepared from the Indian seaweeds was assessed at Pasteur Institute of Epidemiology and Microbiology, St' Petersburg, Russia. The following protocol was observed:

A—Preparation of Influenza Virus Titer

Stock solution of Influenza viral strain subtype A (Mississippi/1/85) was diluted in seven test tubes with peptonic medium in descending order to achieve virus concentrations from 10⁻² to 10⁻⁷ for Example-1 and for subtype B (B/Jamanashi/1/166/95) diluted in seven test tubes to achieve virus concentrations from 10⁻² to 10⁻⁷ for Example-2.

B—Preparation of Peptonic Medium

Gelatin (2.0 g) was thoroughly mixed in 100 ml double distilled water. This was followed by addition of NaCl (8.0 g), KCl (0.6 g), CaCl₂ (0.8 g), MgCl₂ (0.15 g), glucose (0.9 g) and 25 ml of phenol (0.01%). The volume of the solution was made up to 1000 ml with double distilled water. 0.1 g of antibiotic (Penicillin: 1000 units/ml) was also added and pH of the solution was maintained between 6.5 and 7.0. The solution was sterilized in an autoclave for one hour at 15 lbs. The pH of the medium was adjusted with NaHCO₃ to achieve a pH of 6.0 before the experiment.

C—Preparation of Extracts

0.5 ml of extract prepared from each species of seaweeds was transferred to 4 test tubes (marked as Ni, N₂, N₃, N₄) each containing 4.5 ml of peptonic solution to achieve a dilution of 1/10 times.

### Experiment-1

### Experiment with Chorio-Allantoic Membrane (CAM) of Chick Embryo for in vitro Studies and Viral Strain Subtype A (Mississippi/1/85)

Two sets of multiwell plates were thoroughly cleaned with soap and washed several times with double distilled water and then with alcohol for sterilization. In each well of the multiwell plates, 0.5 ml of peptonic medium was transferred with a sterilized pipette. Two fertilized eggs of chicken (13 days old) were cleaned with freshwater to remove all impurities. The eggs were also cleaned thoroughly with absolute alcohol. A small cut was made at the narrow end of the eggs with the help of a pair of sharp scissors and shell piece was removed. The egg with growing embryo was emptied in a glass bowl. Small pieces of the shell (5×5 mm) with chorio-allantoic membrane (CAM) were cut and transferred immediately into the peptonic medium. A single piece of the shell was transferred in the well of each multiwell plate so as to completely dip in the peptonic medium.

Multiwell plate I: The multiwell plate-I was divided into three groups with four wells in each column and 6 wells in each row. 0.1 ml of extract (1/10) prepared from Spatoglossum asperum was pipetted from N, test tube and transferred in all wells of N-column. For N₂-column, 0.1 ml of extract (1/10) prepared from Padina tetrastromatica was pipetted from the test tube N₂ and transferred into all wells of N₂-column.

The third column was identified as control preparation column where extract prepared four seaweeds were transferred serially as in first row of four wells solution from N₁ test tube, second row from N₂ test tube and so on to fourth row where solution from N₄ test tube was added. The multiwell plate-I was finally closed properly and kept at a constant temperature of 34±1° C. for 2 hours.

Multiwell plate II: The multiwell plate-II was divided into three groups with four wells in each column and 6 wells in each row. 0.1 ml of extract (1/10) prepared from Sargassum tenerrimum was pipetted from N₃ test tube and transferred in all wells of N₃-column. For N₄-column, 0.1 ml of extract (1/10) prepared from Stoechospermum marginatum was pipetted from the test tube N₄ and transferred into all wells of N₄-column. The third column was identified as control virus column where extract prepared different species was not transferred. The multiwell plate-II was finally closed properly and kept at a constant temperature of 34±1° C. for 2 hours.

Both the multiwell plates were removed from oven after 2 hours and in each well of column N₁ to N₄ of plates I and II, 0.1 ml of influenza virus was added in the descending dilution i.e. from 1^{st} row (10⁻²) to 6^{th} row (10⁻⁷). The virus was not added in the wells of control preparation column of plate I. All the two plates were again closed properly and kept at a constant temperature of 36±1° C. for incubation for 48 hours.

The multiwell plates were removed from the oven after 48 hours and shell pieces taken out carefully with the help of fine tweezers avoiding the mixing of the solution of the wells. In each well of all the two plates including the control columns 0.05 ml of chicken erythrocyte (5%) was added with the help of a pipette. Both the plates were kept undisturbed at room temperature for 20-30 minutes and then assessed for antiviral activity by haem-agglutination reaction (HAR). Maximum antiviral activity against viral strain A (Mississippi/1/85) was observed with extract prepared from Sargassum tenerrimum. The antiviral activity was moderate in Padina tetrastromatica (Table 1).

TABLE 1
Results of the neutralization reaction to screen antiviral activity using
fragments of chicken embryo (CAM) and influenza viral strain subtype A
(Mississippi/1/85)
Virus dilution (A/Mississippi/1/85)

| | | | | | | | EID₅₀ | Percent |
|---|---|---|---|---|---|---|---|---|
| Sample No | 10⁻² | 10⁻³ | 10⁻⁴ | 10⁻⁵ | 10⁻⁶ | 10⁻⁷ | (lg) | inhibition |
| Control virus | ++ | ++ | ++ | ++ | −− | −− | 5.50* | — |
| | ++ | ++ | ++ | ++ | −− | −− | | |
| S. asperum | ++ | ++ | + | −− | −− | −− | 4.50* | 18.8 |
| | ++ | ++ | + | −− | −− | −− | | |
| | | | + | | | | | |
| | | | + | | | | | |
| P. tetrastromatica | ++ | ++ | −− | −− | −− | −− | 3.25* | 40.9 |
| | ++ | ++ | −− | −− | −− | −− | | |
| S. tenerrimum | ++ | ++ | −− | −− | −− | −− | 3.00* | 45.4 |
| | ++ | ++ | −− | −− | −− | −− | | |
| S. marginatum | ++ | ++ | +− | −− | −− | −− | 3.75* | 31.8 |
| | ++ | ++ | −− | −− | −− | −− | | |

+ Positive shows the presence of virus
− Negative shows the absence of virus
*0.5 added to each value

### Experiment-II

### Experiment with Chorio-Allantoic Membrane (CAM) of Chick Embryo for in vitro Studies and Viral Strain Subtype B (B/Jamanashi/1/166/95)

The protocol for further confirming the antiviral activity in the extract prepared from the seaweeds was same as described in Experiment-1. Maximum antiviral activity against viral strain B (B/Jamanashi/1/166/95) was observed in the extract prepared from S. marginatum that was followed by in S. asperum and P. tetrastromatica. The antiviral activity was moderate in S. tenerrimum (Table 2).

TABLE 2
Results of the neutralization reaction to screen antiviral activity using
fragments of chicken embryo (CAM) and influenza viral strain subtype B
(B/Jamanashi/1/166/95)
Virus dilution (B/Jamanashi/1/166/95)

| | | | | | | | EID₅₀ | Percent |
|---|---|---|---|---|---|---|---|---|
| Sample No | 10⁻² | 10⁻³ | 10⁻⁴ | 10⁻⁵ | 10⁻⁶ | 10⁻⁷ | (lg) | inhibition |
| Control virus | ++ | ++ | ++ | ++ | −− | −− | 5.00* | — |
| | ++ | ++ | ++ | −− | −− | −− | | |
| S. asperum | ++ | −− | −− | −− | −− | −− | 2.50* | 50.0 |
| | ++ | −− | −− | −− | −− | −− | | |
| P. tetrastromatica | ++ | −− | −− | −− | −− | −− | 2.50* | 50.0 |
| | ++ | −− | −− | −− | −− | −− | | |
| S. tenerrimum | ++ | ++ | −− | −− | −− | −− | 3.00* | 40.0 |
| | ++ | −− | −− | −− | −− | −− | | |
| S. marginatum | ++ | −− | −− | −− | −− | −− | 2.00* | 60.0 |
| | ++ | −− | −− | −− | −− | −− | | |

+ Positive shows the presence of virus
− Negative shows the absence of virus
*0.5 added to each value

The main advantages of the present invention is to identify antiviral activity against influenza virus in the extract prepared from Indian seaweeds.

The above description must be understood as being illustrative of the present invention and various modifications are possible without departing from the scope of the invention in any manner.

We claim:

1. An extract with antiviral activity comprising a mixture of extracts from Spatoglossum asperum., Padina tetrastromatica, Sargassum tenerrimum and Stoechospermum marginatum.

2. A process for the preparation of an extract with antiviral activity from seaweeds Spatoglossum asperum., Padina tetrastromatica, Sargassum tenerrimum and Stoechospermum marginatum comprising cleaning the seaweeds with filtered seawater, grinding the seaweeds with double distilled water to obtain a thick paste, lyophlising the paste and then dissolving the lyophilized paste in double distilled water, dried protosubtiline enzyme to the solution and then fermenting the mixture, preparing an active extract by adding concentrated HCl and digesting the material, reducing the pH of the solution to 5.6 pH, isolating the active extract by keeping the resultant solution in a separating flask and removing the middle part of the solution after the formation of different layers.

3. A process as claimed in claim 2 wherein cleaning of the weeds is done with filtered seawater (<5 micron).

4. A process as claimed in claim 2 wherein the grinding of the seaweeds is done in a mixer with double distilled water for making a thick paste.

5. A process as claimed in claim 2 wherein the lypholization of the mixture is done by a table top freeze drying unit for 8 hours.

6. A process as claimed in claim 2 wherein the active extract is prepared by dissolving 500 g of lypholized seaweed in 125 ml of double distilled water in a double necked round bottom distillation flask.

7. A process as claimed in claim 2 wherein the active extract is prepared by adding 10 g dried protosubtiline enzyme and fermenting it at a constant temperature of 40° C. for 2 hours.

8. A process as claimed in claim 2 wherein the active extract is prepared by adding 175 ml of Conc HCl.

9. A process as claimed in claim 2 wherein the digestion of the material is done for 20 hours at a constant temperature of 100±2° C. on a heating mantle.

10. A process as claimed in claim 2 wherein the pH of the solution was achieved to 5.6 pH by adding sodium hydroxide.

11. A process as claimed in claim 2 wherein the active extract is isolated by keeping the resultant extract in a separating flask for 10 days and carefully removing the middle part of the solution after the formation of different layers.

12. A process as claimed in claim 2 wherein the digestion and distillation of the fermented solution is done with concentrated hydrochloric acid (12-15% of the total weight) at a temperature of 90-105° C. for a period ranging between 12 and 20 hours.

13. A process as claimed in claim 2 wherein the pH of the extract is reduced using an alkali selected from the group consisting of NaOH, KOH and NH₃OH after cooling the resultant solution at room temperature.

14. An antiviral formulation comprising a mixture of extracts from Spatoglossum asperum., Padina tetrastromatica, Sargassum tenerrimum and Stoechospermum marginatum along with pharmaceutically acceptable additives.

15. A method for the treatment of influenza comprising administering to a patient suffering from influenza, an antiviral formulation comprising a mixture of extracts from Spatoglossum asperum., Padina tetrastromatica, Sargassum tenerrimum and Stoechospermum marginatum along with pharmaceutically acceptable additives.

16. A method as claimed in claim 15 wherein the subject is a mammal.

17. A method as claimed in claim 15 wherein the mammal is a human being.
